(19) United States
(12) Patent Application Publication
(10) Pub. No.: US 2004/0131198 A1
(43) Pub. Date: Jul. 8, 2004

(54) Noise measurement apparatus and noise measuring cable

(75) Inventors: Yoshiyuki Watanabe, Chiba (JP); Masahiro Okazaki, Kanagawa (JP); Kouichirou Iwanaga, Tokyo (JP)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Sony Corporation

(21) Appl. No.: 10/737,597

(22) Filed: Dec. 16, 2003

(30) Foreign Application Priority Data
Dec. 24, 2002 (JP) P2002-372120

(51) Int. Cl. A61F 11/06; G10K 11/16; H04B 15/00; H03B 29/00

(52) U.S. Cl. 381/71.7; 381/94.1

(57) ABSTRACT

A noise measurement apparatus capable of performing accurate measurement stably without fluctuation and a noise measuring cable to be used for the apparatus are provided. The noise measuring cable, which connects a measurement object to a connector electrically, includes an insulation tube and a conductive wire mesh covering the insulation tube. Extended portions formed by extending the wire mesh from both the end portions of the insulation tube to narrow are formed. The tip of the extended portion on one side is connected to a connector and resistor side, and a clip which is freely attached to and detached from the measurement object is connected to the tip of the extended portion on the other side.

### CROSS REFERENCE TO RELATED APPLICATIONS

The present document is based on Japanese Priority Document JP2002-372120, filed in the Japanese Patent Office on Dec. 24, 2002, the entire contents of which being incorporated herein by reference.

### BACKGROUND OF THE INVENTION

1. Field of the Invention

The present invention relates to a noise measurement apparatus for measuring a noise that is generated when that measurement object is placed an operative state of a measurement object housed in a hermetically sealed conductive housing, and to a noise measuring cable to be used for the measurement apparatus. More particularly, the present invention relates to a noise measurement apparatus for measuring a common mode noise generated at a position between a reference plane of a housing and a measurement object, and relates to a noise measuring cable to be used for the apparatus.

2. Description of the Related Art

For measuring a radiation noise of electrical and electronic equipment, it has been conventionally ordinary to measure remote field intensity at a specific electromagnetic environment measuring place called as an anechoic chamber or an open site, or to use a comparatively large-sized simple measurement apparatus.

Moreover, International Electrotechnical Commission (IEC) Standard examines Work Bench Faraday Cage Method for making it possible to measure common mode noise simply without using any large-scale measurement field such as the anechoic chamber or the like.

The Work Bench Faraday Cage Method is a technique for measuring the common mode noise, which is generated at a position between a metal case and a test substrate, by placing the test substrate at a position 30 mm away from the bottom face of the metal case and keeping the impedance of the test substrate constant.

The common mode noise is the noise forming the same directional voltages or current components on two power supply wires or signal wires. The common mode noise is generated when mismatching of the impedance exists between a noise source and a transmission line, and the return circuit of the generated common mode noise is a housing or the ground as a passage.

Because the common mode noise flows to the ground or a ground wire, the common mode noise causes conducted disturbance to other equipment using the ground or the ground wire commonly. Moreover, a noise current flows through a large loop circuit including a conduction wire and a reference plane when the common mode noise is generated, and consequently the noise current generates an electromagnetic wave. Hence, the electromagnetic wave may easily cause a radiation hazard to adjacent other equipment.

There is an apparatus disclosed in, for example, Patent Document 1 as an apparatus capable of measuring the common mode noise, which is regarded as being important for a measure to counter noise by applying Work Bench Faraday Cage Method. The apparatus does not need an antenna and measurement attachments, which are essential for measurement in an anechoic chamber, and the whole system of the apparatus can be located on a table. Consequently, space-saving designing can be achieved.

Patent Document 1: Japanese Patent Application Publication No. 2002-181863

Moreover, Patent Document 1 discloses a noise measuring cable 60 which is shown in FIG. 20 and is composed of an insulation tube 61 and a wire mesh 62 covering the insulation tube 61 as a noise measuring cable to be used in the apparatus.

On both ends of the noise measuring cable 60, copper tapes 66 are wound over the wire mesh 62. Four leads 64b of four resistors 64 each having the resistance of 390 Ω are soldered to the copper tape 66 on one end side (the left end side in FIG. 20) (see FIG. 21). The leads of the four resistors 64 on the other side are collected to be one lead, and the collected lead 64a is soldered to a connector 7. The four resistors 64 are connected in parallel between the connector 7 and one end side of the noise measuring cable 60. The connector 7 is attached to a side wall portion 2c of a metallic housing 2.

The connection chip portion 65a of an alligator clip 65 is connected to the copper tapes 66 on the other side of the noise measuring cable 60. Incidentally, in FIG. 20, a reference letter L2 designates the length of the insulation tube 61 and the wire mesh 62 covering the insulation tube 61. A reference letter L1 designates the length of the resistors 64 (including the length of the leads) lying between the one end of the noise measuring cable 60 and the connector 7. A reference letter L3 designates the length of the connection chip portion 65a of the alligator clip 65.

A measurement object (not shown) is located in the metallic housing 2, and a measurement portion of the measurement object is nipped by the alligator clip 65 of the noise measuring cable 60. By the use of the noise measuring cable 60, the common mode noise during the operation of the measurement object is measured with a spectrum analyzer or the like installed on the outside of the housing 2 through the resistors 64 and the connector 7.

### SUMMARY OF THE INVENTION

When the present inventors took measurements using a conventional noise measurement apparatus, a problem arose where measurement results fluctuated. The inventors thus come to believe that the following could be the causes thereof.

Even a small inductance, which does not normally matter at a low frequency circuit, causes the increase of impedance when the frequency becomes higher. Consequently, it can be considered that in a high frequency common mode noise, the inductances of the leads 64a and 64b of the resistors 64, and of the metallic connection chip portion 65a of the alligator clip 65 cannot be neglected, and that the frequency characteristic of the impedance of the noise measuring cable 60 is deteriorated by the inductances.

In Work Bench Faraday Cage Method, the electric field intensity is calculated on the prior condition that the noise measuring cable 60 has a fixed frequency characteristic of impedance. Consequently, an unstable frequency characteristic of impedance causes an error of electric field intensity, which is calculated on the basis of the impedance.

The present invention was made in view of the above-mentioned problems. The present invention aims to provide a noise measurement apparatus capable of performing accurate measurement stably without any fluctuation and a noise measuring cable to be used for the apparatus.

In a noise measurement apparatus of the present invention, a noise measuring cable capable of leading a noise, which is generated from a measurement object housed in a conductive housing, to the outside of the housing includes an insulation tube, a conductive wire mesh covering the surface of the insulation tube, and extended portions formed by extending the wire mesh from both the end portions of the insulation tube and then narrowing the extended wire mesh.

A noise measuring cable of the present invention includes an insulation tube, a conductive wire mesh covering the surface of the insulation tube, and extended portions formed by extending the wire mesh from both the end portions of the insulation tube to narrow.

In the related art, a noise measuring cable is configured to connect the leads of resistors and a connection chip portion of an alligator clip to copper tapes wound over the end portions of an insulation tube. The conventional configuration can be considered to aim to secure easy and stable soldering connection of the leads and the connection chip portion to the copper tapes. The configuration makes, however, the leads of the resistors and the connection chip portion longer than necessary lengths.

On the contrary, in the present invention, the wire mesh is extended from both the end portions of the insulation tube to narrow to form the extended portions, and a connection object such as a resistor, a connector and the like is connected to the tip of one of the extended portions.

That is, the present invention adopts the structure in which the leads of the resistors and the connection chip portion of an alligator clip or the like are replaced by the extended portions of the wire mesh. Generally, the inductance of a mesh-shaped conductor decreases especially when the mesh size is formed to be small, and thereby higher frequency electromagnetic events can be suppressed more effectively.

The extended portions have the shapes in which the extent of the portions is narrowed as the position of the extent becomes farther from both the end portions of the insulation tube. As examples of such a shape, a substantial cone, or a substantial triangle in case of being viewed on a plane can be cited.

As examples of the connection object for the noise measuring cable, a resistor, a connector, a spectrum analyzer, a voltage measurement device, a current measurement device, an amplifier, a termination resistor, a computer and the like can be cited.

For example, a connector being one of the connection objects is provided on a wall portion of a housing (the wall portion is not limited to a side wall portion, but may be a top wall portion or a bottom wall portion). The noise measuring cable is connected to an electrode facing to the inside of the housing through a resistor. Otherwise, when a resistor is built in a connector, the noise measuring cable is directly connected to the resistor. In this connector, a spectrum analyzer, a high frequency volt meter, an amplifier, a termination resistor, and the like, which are arranged on the outside of the housing, are connected to an electrode facing to the outside of the housing. Consequently, a measurement object which is located in the housing and connected to the connector through the noise measuring cable, is electrically connected to the various connection objects arranged on the outside of the housing through the connector and the resistor.

Generally, there is stray impedance such as electric capacity between a conduction wire and a reference plane. The influence of the stray impedance cannot be neglected at a high frequency. Consequently, to cover the lead of the resistor connected to the noise measuring cable with an insulating resin is effective for suppressing the stray impedance.

Otherwise, a chip resistor may be used as the resistor connected to the noise measuring cable, and the chip resistor maybe built into the connector. A surface mounting leadless chip resistor has a structure in which a resistance element (for example, made by mixing a material to be resistance with glass and by sintering the mixture) in a thick film or a thin film is formed on, for example, a ceramic matrix, and a central resistance element is disposed between electrodes on both ends, differently from a resister having lead wires (such as a carbon coating resistor, a metal coating resistor, a solid resistor or the like). The chip resistor has no spiral groove, and thereby it is considered to be advantageous in use at a high frequency.

Moreover, when one of the extended portions of the wire mesh is connected to a connection object such as a resistor, a connector or the like with a connection tool which can be freely attached or detached, attachment or detachment operations of a noise measuring cable to and from the resistor or the connector can be easily performed, and the durability of the solder connection portion of the wire mesh and the resistor or the connector can be improved.

As the connection tool which can be freely attached or detached, for example, an alligator clip, an IC clip (also called as a micro clip or a nano clip) and the like having a structure capable of nipping the connection portion of the measurement object such as the resistor, the connector or the like, or the connection portion of the measurement object can be cited. The alligator clip includes two metal pieces, one end side of which is used as a fulcrum and the other end side of which is opened or closed, and the alligator clip nips a connection portion on the other end side. The IC clip includes two metal thin lines the tip portions of which are opened or closed, and the IC clip can nip a connection portion with the metal thin lines by entering the metal thin lines into even a minute gap into which the metal pieces of the alligator clip cannot enter. Consequently, the IC clip is advantageous as a detachable connection tool for connecting to a measurement object of form with which there often are spatial limitations around the connective portion.

In addition, a configuration in which a tape capable of being exfoliated (both types being conductive or insulative applicable) is used as a connection tool to attach the tip portions of the extended portions of a wire mesh to the connection object or the measurement object may be adopted.

The resistor may be one or plural. Moreover, in case of being plural, the resistors may be connected in series or in parallel. The kinds and the number of the resistors to be used are suitably selected in order that the frequency characteristic of impedance may be the optimum according to a measurement frequency band.

As described above, according to the present invention, the noise measuring cable in which the extended portions are formed by extending the wire mesh from both the end portions of the insulation tube and by then narrowing them is formed, whereby the frequency characteristic of the impedance of the noise measuring cable can be consequently stabilized. Therefore, accurate noise measurement without any fluctuation can be performed.

### BRIEF DESCRIPTION OF THE DRAWINGS

FIG. 1 is a perspective view of a noise measurement apparatus according to an embodiment of the present invention;

FIG. 2 is a plan view of the noise measurement apparatus;

FIG. 3 is a block diagram showing a configuration of a noise measurement system using the noise measurement apparatus;

FIG. 4 is a block diagram showing a configuration of a system for measuring a frequency characteristic of a impedance of a noise measuring cable;

FIG. 5 is a side sectional view of the principal part of a noise measurement apparatus according to a first embodiment;

FIG. 6 is a side sectional view of the principal part of a noise measurement apparatus according to a third embodiment;

FIG. 7 is a side sectional view of the principal part of a noise measurement apparatus of a fourth embodiment;

FIG. 8 is a sectional view of a connector and resistors attached to the connector in the noise measurement apparatus of the first embodiment;

FIG. 9 is a sectional view of a connector and a resistor attached to the connector of the noise measurement apparatus of the third embodiment;

FIG. 10 is a sectional view of a connector and a resistor built in the connector of the noise measurement apparatus of a fourth embodiment;

FIG. 11 is a side sectional view of the principal part of a noise measurement apparatus according to a fifth embodiment;

FIG. 12 is a side sectional view of the principal part of a noise measurement apparatus of a sixth embodiment;

FIG. 13 is a side sectional view of the principal part of a noise measurement apparatus of a seventh embodiment;

FIG. 14 is a perspective view of the principal part of a noise measurement apparatus of an eighth embodiment;

FIG. 15 is a sectional view taken along a line [15]-[15] in FIG. 5;

FIG. 16 is a graph showing an example of comparison between frequency characteristics of the impedance of noise measuring cables of the related art and the second embodiment;

FIG. 17 is a graph showing an example of comparison between frequency characteristics of the impedance of noise measuring cables of the first embodiment and of the third embodiment;

FIG. 18 is an enlarged view of a part of the graph of FIG. 17;

FIG. 19 is a graph showing an example of comparison among frequency characteristics of the voltage standing wave ratios (VSWR's) of noise measuring cables of the related art, of the first embodiment and of the third embodiment;

FIG. 20 is a side sectional view of the principal part of the noise measurement apparatus of the related art; and

FIG. 21 is an enlarged perspective view showing the principal part of FIG. 20.

### DETAILED DESCRIPTION OF THE PREFERRED EMBODIMENTS

In the following, the attached drawings are referred to while the preferred embodiments of the present invention are described.

(First Embodiment)

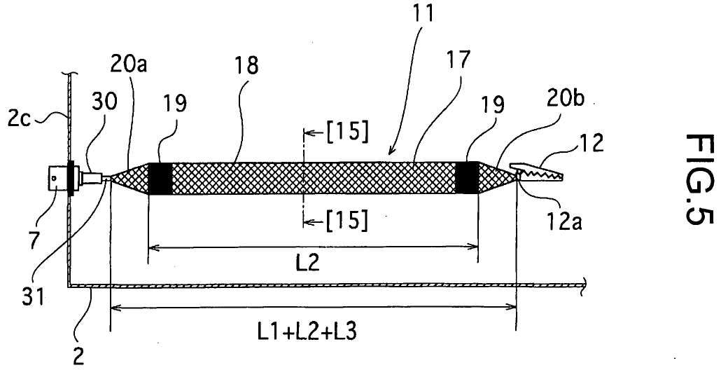

FIG. 5 shows a side view of a noise measuring cable 11 according to a first embodiment. Incidentally, the state shown in FIG. 5 is the state in which one end side of the noise measuring cable 11 is connected to a connector 7 through resistors (built in a resistance enclosure 30). Moreover, a sectional view taken along a line [15]-[15] in FIG. 5 is shown as FIG. 15.

The noise measuring cable 11 is mainly composed of a insulation tube 17 and a wire mesh 18 covering the surface of the insulation tube 17. The insulation tube 17 has flexibility, and made of, for example, a rubber material. The wire mesh 18 is made by weaving many metal slender wires. The insulation tube 17 extends only within the range of a length designated by a letter L2 in FIG. 5, and the thickness (the internal diameter and the external diameter) of the insulation tube 17 is equal all over the range of the length L2.

Extended portions 20a and 20b, which are extended from both the end portions of the insulation tube 17, are formed in the wire mesh 18. The extended portions 20a and 20b are narrowed toward their extension directions. That is, the extended portions 20a and 20b have tapered shapes in which extents gradually become narrower as distances from both the end portions of the insulation tube 17 become larger. The insulation tube 17 does not extend in the insides of the extended portions 20a and 20b.

Insulating tapes (for example, resin-made tapes) 19 are wound over the wire mesh 18 at the both the end portions of the insulation tube 17 so that the wire mesh 18 is fixed to the insulation tube 17. Consequently, the insulating tapes 19 prevent the wire mesh 18 from coming loose to keep the shapes of the extended portions 20a and 20b stably.

The tip portion of the extended portion 20a on one end side of the noise measuring cable 11 (on the left side in FIG. 5) is connected to a connection terminal 31 by, for example, being soldered. The connection terminal 31 is electrically connected to the connector 7 attached to a side wall portion 2c of a housing 2 through resistors built in the resistance enclosure 30, as it will be described later.

The tip portion of the extended portion 20b on the other side of the noise measuring cable 11 (on the right side in FIG. 5) is connected to a metallic alligator clip 12 by, for example, being soldered. The alligator clip 12 functions as a connection tool for connecting the noise measuring cable 11 to a measurement object electrically by nipping a measurement portion of the measurement object, which will be described later.

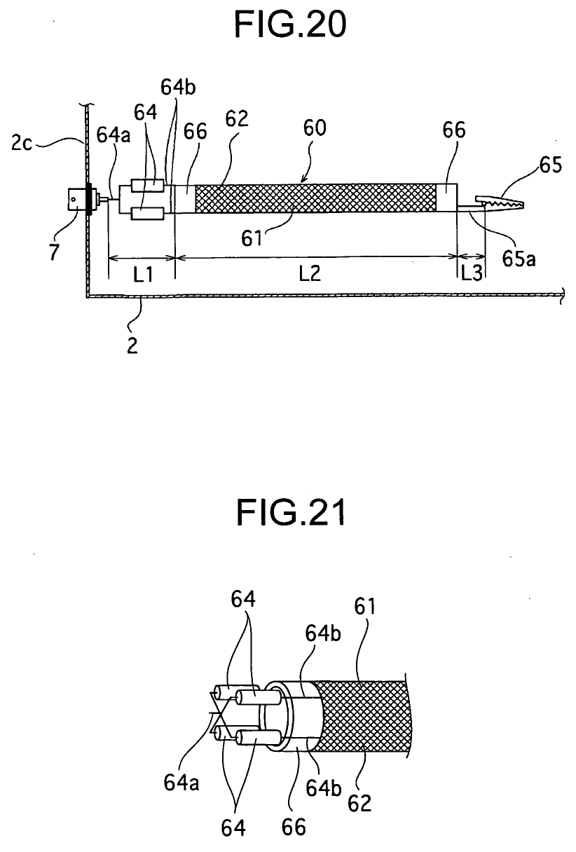

Next, the length of the noise measuring cable 11 of the present embodiment is compared with that of the conventional noise measuring cable 60, which has been described with reference to FIG. 20 while the length of the present embodiment is described.

In the conventional noise measuring cable 60, the wire mesh 62 has the same length L2 as that of the insulation tube 61 (the wire mesh 62 also covers the insulation tube 61 under the copper tapes 66 in FIG. 20). On the contrary, in the present embodiment shown in FIG. 5, the insulation tube 17 itself has the same length L2 (for example, about 10 cm) as that of the conventional noise measuring cable 60. However, the wire mesh 18 extends from both the end portions of the insulation tube 17 having the length L2. When the extended portions 20a and 20b are added to the length L2, the total length of the wire mesh 18 is a length (L1+L2+L3).

That is, in the conventional noise measuring cable 60 shown in FIG. 20, the length L1 of the resistors 64 (including the leads 64a and 64b) and the portion of the length L3 of the connection chip portion 65a of the alligator clip 65 are replaced by the extended portion 20a and 20b, which have the tapered shapes, of the wire mesh 18 in the present embodiment.

Consequently, in the present embodiment, the leads of the resistors and the connection chip portion 12a of the alligator clip 12 are electrically connected to the wire mesh 18 in the state that the leads and the connection chip portion 12a are as short as possible. Consequently, it is possible to suppress the influence of the inductances of the leads of the resistors and the connection chip portion 12a of the alligator clip 12 to the impedance of the noise measuring cable 11, and then the impedance of the noise measuring cable 11 can be stabilized. Incidentally, experimental results proving the fact will be described later.

Moreover, in the related art, the electrical connection between the resistors 64 and the wire mesh 62, and the electrical connection between the alligator clip 65 and the wire mesh 62, are established with the copper tapes 66 disposed between each of them. On the contrary, the present embodiment has the structure in which the resistors and the alligator clip 12 are directly connected to the wire mesh 18, and consequently the present embodiment does not need any copper tapes. The fact of no copper tape required can be also considered as one of the causes of stabilizing the impedance in the noise measuring cable 11 of the present embodiment by suppressing the inductances of the copper tapes and the stray capacitance between the copper tapes and the housing 2.

Moreover, because in the present embodiment, the wire mesh 18 is fixed to the insulation tube 17 by the use of the flexible and elastic resin-made tapes 19 in place of the copper tapes, the mutual adhering force between the wire mesh 18 and the insulation tube 17 can be heightened. Thus, even if the noise measuring cable 11 is repeatedly used, it is possible to prevent that the wire mesh 18 comes loose, and that the shapes of the extended portions 20a and 20b are lost, and then the state shown in FIG. 5 can be stably maintained.

Moreover, the durability of the soldered portions to outer forces imposed on the soldered portions can be made to be higher in the structure of the present embodiment, in which the connection terminal 31 and the connection chip portion 12a of the alligator clip 12 are soldered to the extended portions 20a and 20b of the flexible wire mesh 18, than in the conventional structure, in which the leads 64b of the resistors 64 and the connection chip portion 65a of the alligator clip 65 are soldered to the rigid copper tapes 66.

(Second Embodiment)

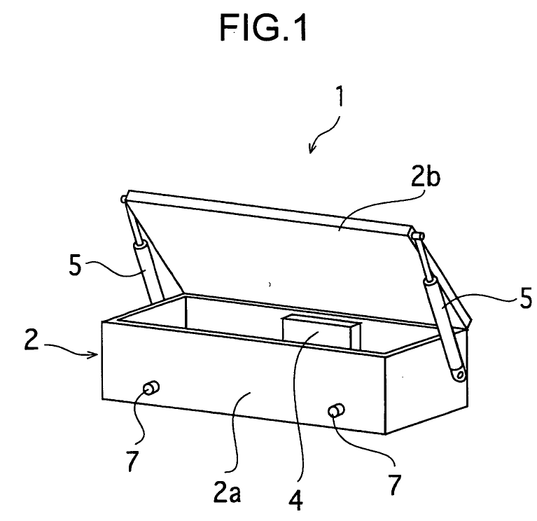

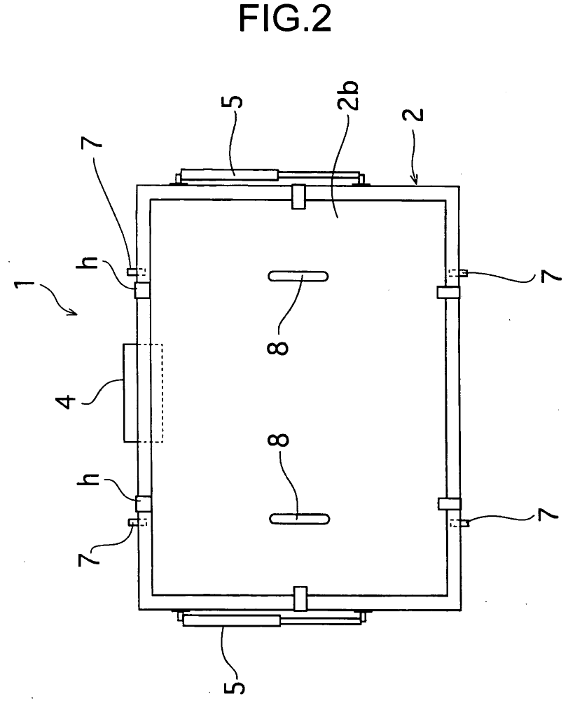

Next, a second embodiment of the present invention will be described. FIG. 1 shows a perspective view of a noise measurement apparatus 1 of the present embodiment, and FIG. 2 shows a plan view thereof.

A metallic housing 2 is composed of a body portion 2a in which a measurement object is placed and a lid portion 2b attached to the body portion 2a in the state of being freely opened and closed with hinges h (see FIG. 2). Shock absorbers 5 configured to include hydraulic dampers are laid between both the side faces of the lid portion 2b and both the side faces of the body portion 2a. The shock absorbers 5 are placed in order to make the opening and closing operations of the lid portion 2b easy to a measurer.

Two connectors 7 are severally provided on the front face side and on the back face side of the body portion 2a. Moreover, a filter box 4, which has electrode terminals for supplying electric power from a power source on the outside of the housing 2 to the measurement object housed in the housing 2, is provided on the back face side.

Handles 8 are provided on the top surface (see FIG. 2) of the lid portion 2b. The measurer holds the handles 8 to open or close the lid portion 2b.

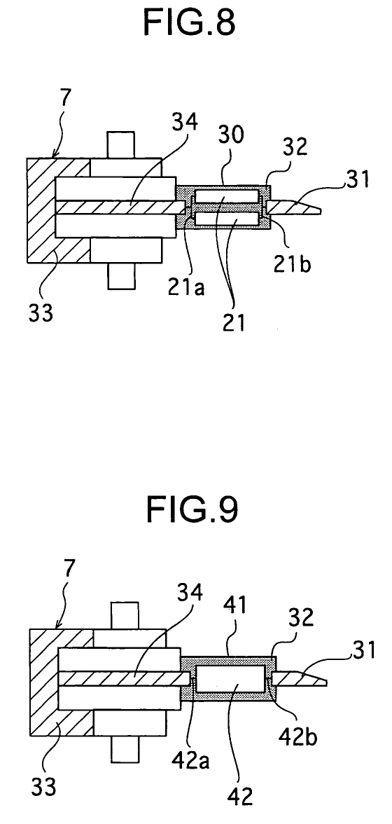

Next, FIG. 8 is referred to while the connectors 7 are described.

The connectors 7 are, for example, Bayonet Neill-Concelman (BNC) connectors. An electrode 34 is included in a connector casing 33. A resistance enclosure (made of an insulating material) 30 is integrally attached to each of the connectors 7.

Four resistors 21 connected in parallel to each other are housed in the resistance enclosure 30. The resistance value of each of the resistors 21 is, for example, 390 Ω.

The resistors 21 are insertion mounting type resistors having leads. Incidentally, the leads 21a and 21b shown in FIG. 8 are showed in the state in which each lead of the four resistors 21 is collected to be one lead. Then, an insulating resin 32 is filled in the resistance enclosure 30 so as to cover the whole resistors 21 including the leads 21a and 21b. A insulating resin 32 suppresses the stray capacitance between the leads 21a and 21b and the housing 2.

The lead 21a on one end side is electrically connected to the electrode 34 of the connector 7 by means of, for example, solder. The lead 21b on the other side is electrically connected to a connection terminal 31 by means of, for example, solder.

The connection terminal 31 projects from the resistance enclosure 30 toward the inside of the housing 2. The tip portion of the extended portion 20a of a noise measuring cable 11 of the first embodiment described above is electrically connected to a connection terminal 31 by mans of, for example, solder. Thus, the noise measuring cable 11 is electrically connected to the connector 7 through the resistors 21. Moreover, the electrode 34 of the connector 7 is electrically connected to an amplifier 13, a spectrum analyzer 14 or a termination resistor 9 on the outside of the housing 2.

Next, the noise measurement of a measurement object using the noise measurement apparatus 1 configured as described above will be described.

The measurement object is a product in the state of incorporating each part finally such as a notebook computer, a digital still camera, a video camera, compact audio equipment, a personal digital assistant (PDA), a portable telephone or the like.

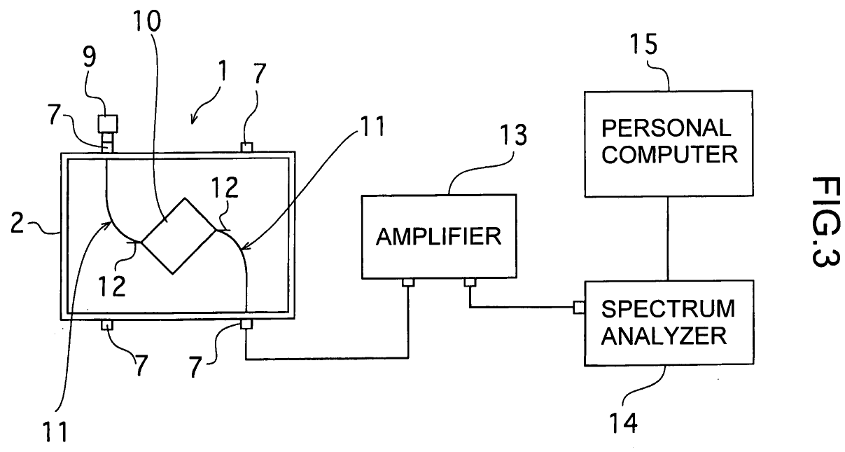

As shown in FIG. 3, a measurement object 10 is housed in the housing 2 of the noise measurement apparatus 1. In the housing 2, two noise measuring cables 11 described above are arranged. The tip portions of the extended portions 20a on one end side of each of the noise measuring cables 11 are severally connected to the connectors 7, which are placed on diagonal positions among the connectors 7, attached on the side wall portions of the housing 2 through the resistors 21 described above.

Alligator clips 12 of the other end sides of each of the noise measuring cable 11 nip measurement portions of the measurement object 10.

On the outside of the housing 2, the termination resistor (for example, 50 Ω) 9 is connected to one of the two connectors 7 to which the noise measuring cables 11 are connected. The high frequency amplifier 13 arranged on the outside of the housing 2 is connected to the other one of the connectors 7.

The amplifier 13 is connected to the spectrum analyzer 14. Moreover, for making it possible to input the measurement results by the spectrum analyzer 14 into the a personal computer 15, the spectrum analyzer 14 is connected to the personal computer 15.

As described above, a loop is formed between the measurement object 10 and the housing 2 through the two noise measuring cables 11, and high frequency common mode noises flowing through the loop are amplified by the amplifier 13 to be derived to the spectrum analyzer 14.

Measurement is performed in the state in which the measurement object 10 is hermetically sealed in the housing 2 by closing the lid portion 2b of the housing 2. In this state, electric power is supplied from the driving power source to the measurement object 10 through a filter box 4, so that the measurement object 10is operated. Then, an electric field intensity U (in dB values) within a predetermined frequency range of the high frequency common mode noises generated at this time is measured by the spectrum analyzer 14.

The measurement values measured by the spectrum analyzer 14 are stored in a hard disk built in the personal computer 15, and the central processing unit of the personal computer 15 performs various kinds of processing such as impedance correction or the like with respect to the measurement values.

The electric field intensity E (in true values) defined by Work Bench Faraday Cage Method is given by the following theoretical formula (1).

E=(7/R)×√{(U²)/150} [dBμV/m] (1)

where the letter R indicates the distance [m] between a measurement object and a measuring antenna in radiated emission measurement; the letter U indicates measurement values [dBμV] to be obtained by the measurement system shown in FIG. 3; and the numeral 150 indicates the impedance [Ω] of the noise measuring cable 11.

As apparent from the formula (1), when the impedance of the noise measuring cable 11 is dispersed, the values of the electric field intensity E are also fluctuated, hence it makes impossible to perform accurate measurement.

In the following, experiments for comparing the frequency characteristic of the impedance of the noise measuring cable 11 according to the embodiment of the present invention with that of a conventional noise measuring cable 60 (see FIG. 20) will be described.

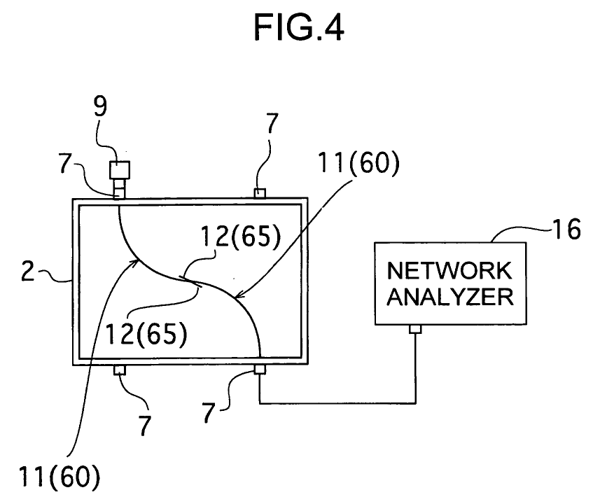

The measurement system of this comparison experiment is shown in FIG. 4. In this measurement, the alligator clips 12 of each of the noise measuring cable 11 are connected to each other by nipping each other. The measurement object 10 is not housed in the housing 2.

The termination resistor 9 is connected to one of the noise measuring cables 11 through one of the connectors 7 on the outside of the housing 2, and the other one of the noise measuring cables 11 is connected to a network analyzer 16 arranged on the outside of the housing 2 through another connector 7.

In such a connection state, the network analyzer 16 measures the frequency characteristics of the impedance of the noise measuring cables 11 in a predetermined frequency range (for example, the range of from 30 MHz to 1 GHz).

The measurement was also performed to the conventional noise measuring cables 60. That is, as shown in FIG. 20, each of the leads 64b of each of the resistors 64 is connected to the copper tape 66 wound over one end side of the noise measuring cable 60, and the lead 64a of each of the resistors 64, which is collected to be one lead, is connected to the connector 7.

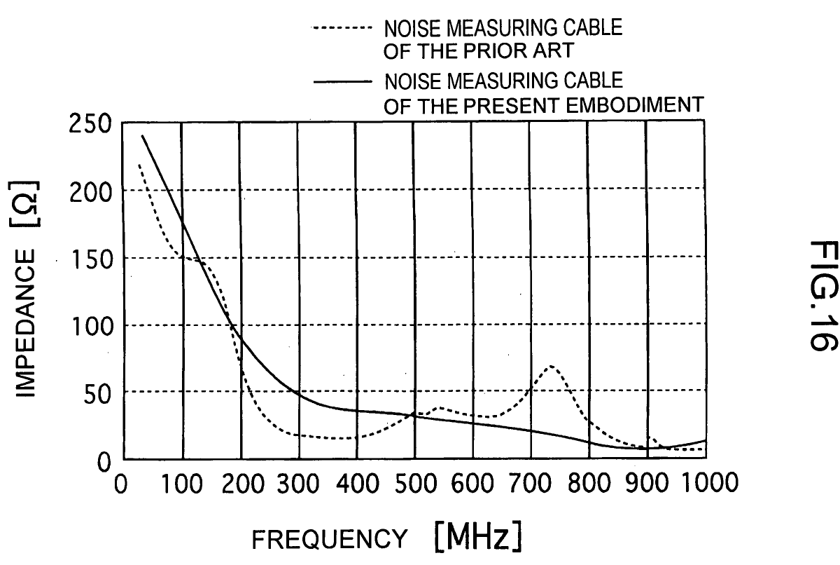

The comparison results are shown in FIG. 16. The abscissa axis indicates frequencies, and the ordinate axis indicates impedance. A dotted line curve indicates the frequency characteristic of the impedance of the conventional noise measuring cables 60, and a full line curve indicates the frequency characteristic of the impedance of the noise measuring cables 11 of the embodiment.

As apparent from the result, the variations of the impedance can be suppressed in the noise measuring cables 11 of the embodiment more than in the conventional noise measuring cables 60 (in particular, the impedance frequency characteristic of the noise measuring cables 11 is improved in the range of from 300 MHz to 1000 MHz), and no resonance phenomena are generated in the noise measuring cables 11.

The stabilization of the impedance of the noise measuring cable 11 brings about the accurate measurement of the electric field intensity E to be obtained from the formula (1). Incidentally, it is ideal that the impedance of the noise measuring cable 11 is stabilized at 150 Ω, but it is difficult to realize the ideal impedance actually. Accordingly, impedance corrections are needed for the electric field intensity E.

However, when the electric field intensity E of the measurement object at the design stage or as unassembled parts stage is measured, it is sufficient to confirm how the electric field intensity E increased or decreased by employing measures to counter noise to the measurement object. Consequently, the impedance corrections are not necessarily needed.

By using the noise measurement apparatus 1 described above, it is possible to judge or estimate the satisfaction of standards related to electromagnetic compatibility (EMC) characteristics at the stage of trial manufacturing or development of a product without bringing the product to an open site or in an anechoic chamber, and consequently the man-hour of measurement and costs can be decreased. Only the measurement for the final standard certification defined strictly in standards or the like is needed to be performed in an open site or an anechoic chamber.

(Third Embodiment)

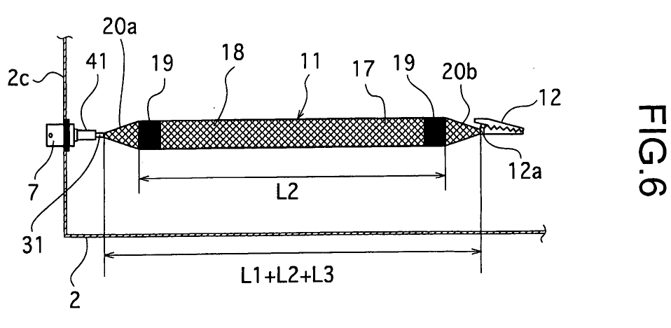

Next, FIGS. 6 and 9 are referred to while a noise measurement apparatus according to a third embodiment is described. Incidentally, the same configuration portions as those of the first and the second embodiments are designated by the same reference numerals as those of the first and the second embodiments, and the descriptions of the same configuration portions are omitted.

The third embodiment differs from the noise measurement apparatus of the second embodiment only in the configurations of the resistors, which are laid between connectors 7 and a noise measuring cables 11.

That is, as shown in FIG. 9, one resistor 42 is housed in a resistance enclosure 41 made of an insulating material. The resistance value of the resistor 42 is, for example, 100 Ω.

The resistor 42 is an insertion mounting type resistor including leads 42a and 42b. Then, an insulating resin 32 is filled in the resistance enclosure 41 so as to cover the whole resistor 42 including the leads 42a and 42b. The stray capacitance between the leads 42a and 42b and a housing 2 is suppressed also in the present embodiment.

The lead 42a on one side is electrically connected to an electrode 34 of each of the connectors 7 by means of, for example, solder, and the lead 42b on the other side is electrically connected to a connection terminal 31 by means of, for example, solder.

The connection terminal 31 projects from the resistance enclosure 41 toward the inside of the housing 2, and the tip portion of the extended portion 20a of the noise measuring cable 11 of the first embodiment is electrically connected to the connection terminal 31 by means of, for example, solder. Thus, the noise measuring cable 11 is electrically connected to one of the connector 7 through the resistor 42. Moreover, the electrode 34 of one of the connectors 7 is electrically connected to the amplifier 13, the spectrum analyzer 14 or the termination resistor 9, which are described above, on the outside of the housing 2.

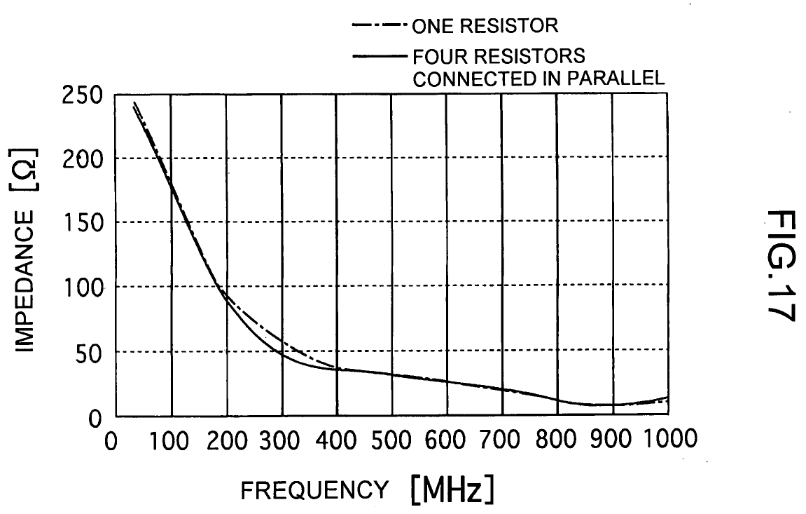

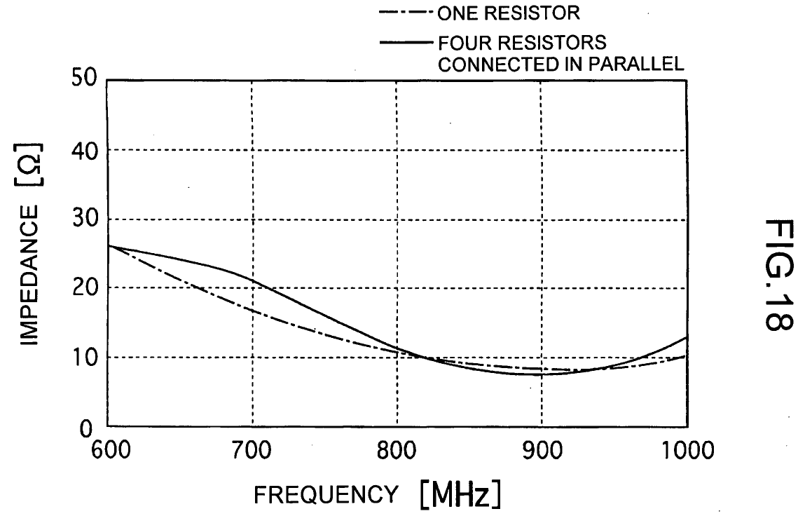

A graph shown in FIG. 17 shows a comparison of the frequency characteristics of impedance of the noise measuring cables 11 in the case of using one resistor 42 of the present embodiment (the form shown in FIG. 9) and in the case of four resistors 21 connected in parallel to each other (the form shown in FIG. 8). Moreover, FIG. 18 shows an enlarged view of the graph of FIG. 17 in the frequency range of from 600 MHz to 1000 MHz. The full line curve indicates the case of four resistors 21, and an alternate long and short dash line curve indicates the case of one resistor 42.

From FIG. 17, in the case of one resistor 42, it is found that the frequency characteristic of impedance in the range of from 200 MHz to 400 MHz is improved (it is preferably that the impedance is stabilized in a region close to a theoretical value of 150 Ω). Moreover, from FIG. 18, in the case of four resistors 21, it is found that the frequency characteristics of impedance in the ranges of from 600 MHz to 800 MHz and in the ranges of from 920 MHz to 1000 MHz are improved.

As described above, because frequency bands, in which the frequency characteristics of impedance are improved, change dependently on the number of resistors or resistance values, the numbers of the resistors or the resistance values are suitably selected according to the frequency band of the common mode noise to be measured.

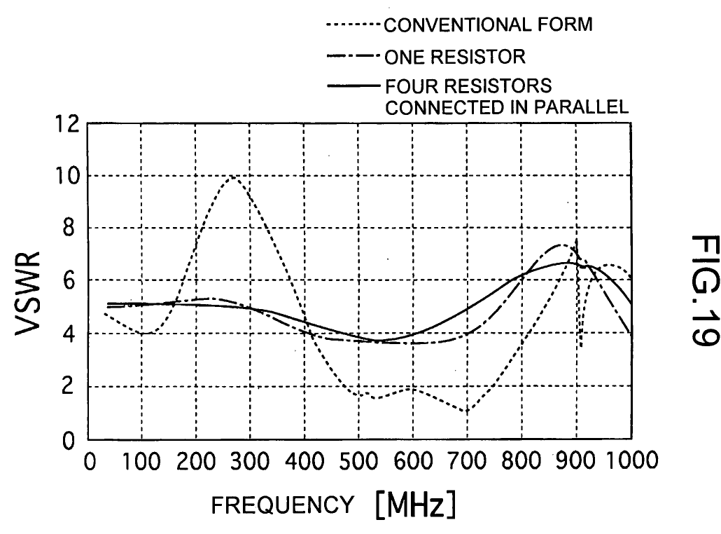

Incidentally, FIG. 19 shows the frequency characteristics of respective voltage standing wave ratios (VSWR's) in the case of the conventional form (the form shown in FIG. 20), the case of one resistor (the form shown in FIG. 9) and the case of four resistors (the form shown in FIG. 8). The VSWR shows a ratio of peak to valley of a voltage amplitude distribution to be generated on a transmission line on which a reflection wave is generated owing to impedance mismatching.

As apparent from this result also, in the embodiments of the present invention, it is possible to suppress variations also in the frequency characteristics of the VSWR's more than those of the reference art to improve the frequency characteristics.

(Fourth Embodiment)

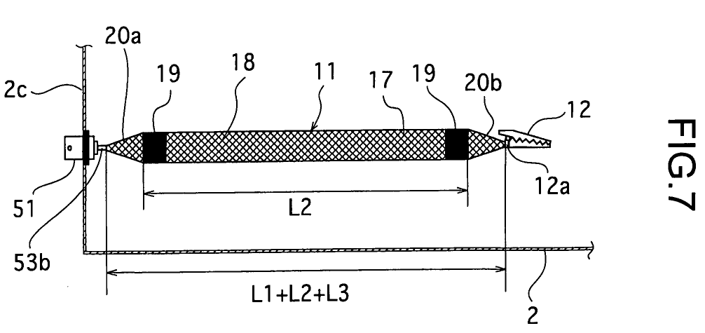

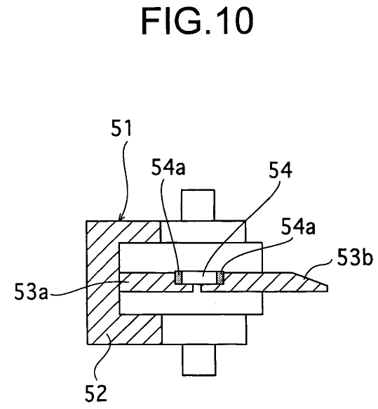

Next, FIGS. 7 and 10 are referred to while a noise measurement apparatus according to a fourth embodiment is described. Incidentally, the same configuration portions as those of the first and the second embodiments are designated by the same reference numerals as those of the first and the second embodiments, and the descriptions of the same configuration portions are omitted.

In the fourth embodiment, as shown in FIG. 10, a chip resistor 54 is used as the resistor, and the chip resistor 54 is built in a connector casing 52 of a connector 51. The other configurations are the same as those of the first and the second embodiments described above.

Both electrodes 54a of the chip resistor 54 are respectively electrically connected to electrodes 53a and 53b of the connector 51 by means of, for example, solder.

The electrode 53b of the connector 51 projects from the connector casing 52 toward the inside of the housing 2. To the electrode 53b, the tip portion of the extended portion 20a of the noise measuring cable 11 of the above-mentioned first embodiment is electrically connected by means of, for example, solder. Thus, the noise measuring cable 11 is electrically connected to the connector 51 through the chip resistor 54. Moreover, the electrode 53a of the connector 51 is electrically connected to the amplifier 13, the spectrum analyzer 14 or the termination resistor 9 on the outside of the housing 2, which are mentioned above.

The chip resistor 54 has no leads and is small in size. The influence of the inductance of the chip resistor 54 to the impedance of a noise measuring cable can be made to be smaller than that of an insertion mounting type resistor having leads. Moreover, because the chip resistor 54 is built in the connector casing 52, the stray capacitance between the housing 2 and the chip resistor 54 can be also suppressed.

(Fifth Embodiment)

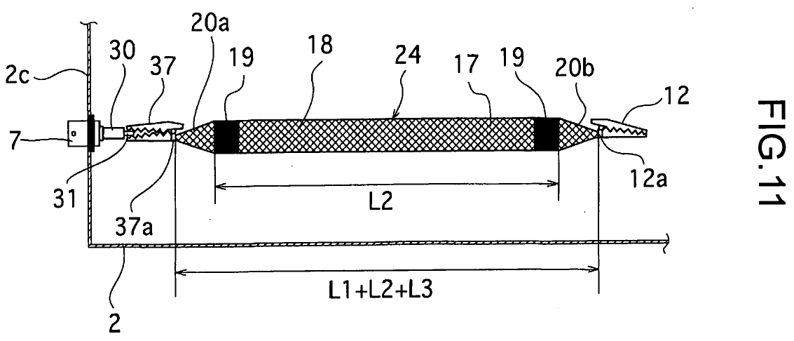

Next, FIG. 11 is referred to while a noise measurement apparatus according to a fifth embodiment is described. Incidentally, the same configuration portions as those of the first and the second embodiments described above are designated by the same reference numerals as those of the first and the second embodiments, and the detailed descriptions of the same configuration portions are omitted.

The present embodiment differs from the first and the second embodiments in that the connection between a noise measuring cable 24 and a resistors 21 (see FIG. 8) built in a resistance enclosure 30 is made to be freely attached or detached.

That is, the tip portion of a connection chip portion 37a of an alligator clip 37 is connected to an extended portion 20a on one end side of a wire mesh 18 by means of, for example, solder. By making the alligator clip 37 nip a connection terminal 31 connected to a resistors 21, the noise measuring cable 24 is electrically connected to one of the connectors 7 through the resistors 21.

By such a configuration, even when excessive external force is acted on one end side of the noise measuring cable 24, or even when external force repeatedly acted though it is small force, the connection between the wire mesh 18 and the alligator clip 37 and the connection between the alligator clip 37 and the connection terminal 31 can be stably maintained. It is needless to say that the attachment or the detachment operation between the noise measuring cable 24 and the connection terminal 31 can be easily performed, and it is easy to deal with a positional change of the connector 7 to which the noise measuring cable 24 is connected. The other configurations and advantages to be obtained are the similar as those of the first and the second embodiments.

(Sixth Embodiment)

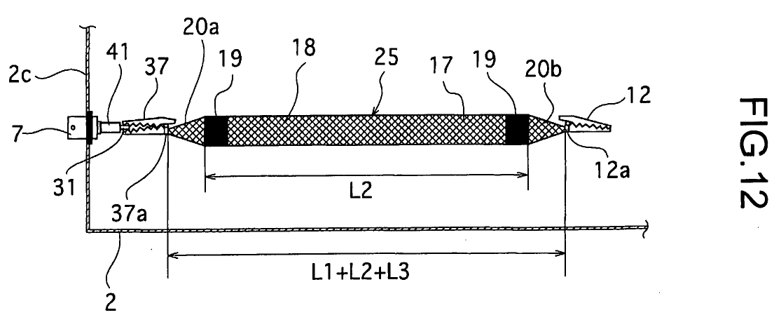

Next, FIG. 12 is referred to while a noise measurement apparatus according to a sixth embodiment is described. Incidentally, the same configuration portions as those of the first, the second and the third embodiments are designated by the same reference numerals as those of the first to the third embodiments, and the detail descriptions of the similar configuration portions are omitted.

The present embodiment differs from the first to the third embodiments in that the connection between a noise measuring cable 25 and a resistor 42 (see FIG. 9) built in a resistance enclosure 41 is made to be freely attached or detached.

That is, a connection chip portion 37a of an alligator clip 37 is connected to the tip portion of an extended portion 20a on one end side of a wire mesh 18 by means of, for example, solder. By making an alligator clip 37 nip a connection terminal 31 connected to the resistor 42, a noise measuring cable 25 is electrically connected to a connector 7 through the resistor 42.

By such a configuration, even when excessive external force is acted on one end side of the noise measuring cable 25, or even when external force repeatedly acted though it is small force, the connection between the wire mesh 18 and the alligator clip 37 and the connection between the alligator clip 37 and the connection terminal 31 can be stably maintained. It is needless to say that the attachment or the detachment operation between the noise measuring cable 25 and the connection terminal 31 can be easily performed, and it is easy to deal with a positional change of the connector 7 to which the noise measuring cable 25 is connected. The other configurations and advantages to be obtained are the similar as those in the first to the third embodiments.

(Seventh Embodiment)

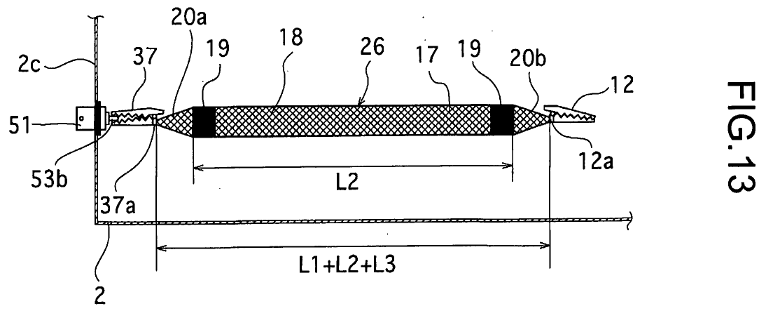

Next, FIG. 13 is referred to while a noise measurement apparatus according to a seventh embodiment is described. Incidentally, the similar configuration portions as those of the first, the second and the fourth embodiments are designated by the same reference numerals as those of the first, the second and the fourth embodiments, and the detailed descriptions of the same configuration portions are omitted.

The present embodiment differs from the first, the second and the fourth embodiments in that the connection between a noise measuring cable 26, and a connector 51 and a chip resistor 54 (see FIG. 10) built in the connector 51 is made to be freely attached or detached.

That is, a connection chip portion 37a of an alligator clip 37 is connected to the tip portion of an extended portion 20a on one end side of a wire mesh 18 by means of, for example, solder. By making the alligator clip 37 nip an electrode 53b of the connector 51, the noise measuring cable 26 is electrically connected to the chip resistor 54 and the connector 51.

By such a configuration, even when excessive external force is acted on one end side of the noise measuring cable 26, or even when external force repeatedly acted though it is small force, the connection between the wire mesh 18 and the alligator clip 37 and the connection between the alligator clip 37 and the electrode 53b of the connector 51 can be stably maintained. It is needless to say that the attachment or detachment operation between the noise measuring cable 26 and the electrode 53b can be easily performed, and it is easy to deal with a positional change of the connector 51 to which the noise measuring cable 26 is connected. The other configurations and advantages to be obtained are the similar as those in the first, the second and the fourth embodiments.

(Eighth Embodiment)

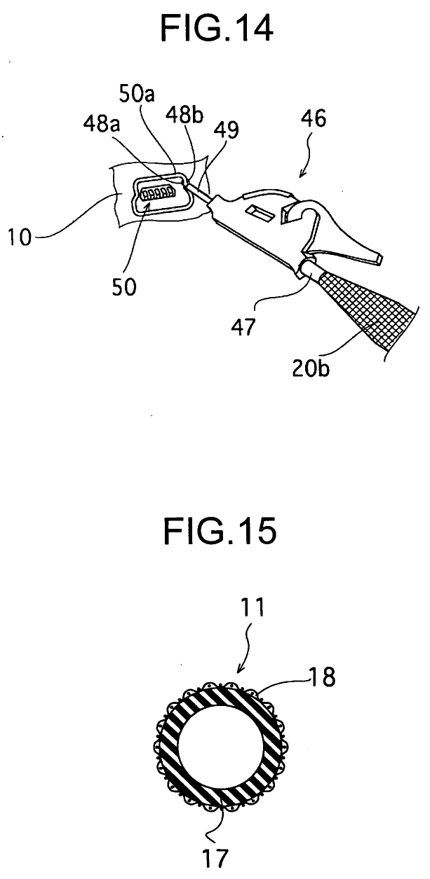

Next, FIG. 14 is referred to while an eighth embodiment is described. Incidentally, the same configuration portions as those of each of the above-described embodiments are designated by the same reference numerals as those of each of the above-described embodiments, and the detailed descriptions of the same configuration portions are omitted.

The present embodiment uses an IC clip 46 as shown in the figure in place of the alligator clip 12 of each of the above-described embodiments.

That is, the tip portion of an extended portion 20b on the other end side of a wire mesh 18 is connected to a lead 47 of the IC clip 46 by means of, for example, solder. The lead 47 is electrically connected to two hook portions 48a and 48b projecting from a cylinder portion 49. The hook portions 48a and 48b nip a conductor portion 50a (for example, the outer frame of a Universal Serial Bus (USB) connector) which gives reference electric potential of a measurement object 10.

By using the IC clip 46, even in case of a minute measurement portion where the alligator clip 12 cannot nip, the measurement portion can be surely connected to the noise measuring cable electrically. The other configurations and advantages to be obtained are the similar as those in each of the above-described embodiments.

Although each of the embodiments of the present invention is described above, it is needless to say that the present invention is not limited to the embodiments. It is possible to change the embodiments variously on the basis of the technical sprit of the present invention.

The connectors 7 are supposed to be the BNC connectors in the embodiments. However, in case of the measurement in which the characteristic at a high frequency band is regarded as important, it is advantages to use a connector having a stable transmission characteristic at the high frequency band. For example, a Sub Miniature Type A (SMA) connector, which is used at a microwave band most popularly, can be cited.

Moreover, the measurement object 10 is not limited to the one in the form of a product in which each component is incorporated finally, but the measurement object 10 may be a substrate, an individual part or a semiconductor device.

Moreover, as long as the influences of the inductance or the stray capacitance can be decreased by making the leads of the resistors 21 and 42 as short as possible, the resistors 21 and 42 are not necessarily covered by the insulating resin 32.

The connection of the connection chip portions 12a, 37a of the alligator clips 12, 37 with the extended portions 20a and 20b of the wire mesh 18 may be performed by coupling the tip portions of the extended portions 20a and 20b to the connection chip portions 12a and 37a.

An electric wave absorber may be provided on the back surface of the lid portion 2b of the housing 2.

Finally, the embodiments and examples described above are only examples of the present invention. It should be noted that the present invention is not restricted only to such embodiments and examples, and various modifications, combinations and sub-combinations in accordance with its design or the like may be made without departing from the scope of the present invention.

What is claimed is:

1. A noise measurement apparatus comprising:
a conductive housing capable of housing a measurement object therein; and
a noise measuring cable capable of leading a noise generated from said measurement object housed in said housing to outside of said housing,
wherein said noise measuring cable comprises an insulation tube, a conductive wire mesh covering a surface of said insulation tube, and extended portions formed by extending said wire mesh from both end portions of said insulation tube and by narrowing said extended wire mesh.

2. The noise measurement apparatus according to claim 1, wherein a connection component, which can be attached to or detached from a connection object, is connected to a tip portion of said extended portion.

3. The noise measurement apparatus according to claim 1, wherein a resistor is connected to a tip portion of one of said extended portions, and said resistor is covered by an insulating resin.

4. The noise measurement apparatus according to claim 1, wherein a connector to which said noise measuring cable can be connected is provided on a wall portion of said housing, and a chip resistor connected to said connector electrically is built in said connector.

5. A noise measuring cable comprising:
an insulation tube, and
a conductive wire mesh covering a surface of said insulation tube,
wherein said cable capable of leading a noise generated from a measurement object housed in a conductive housing to outside of said housing,
wherein said cable comprises extended portions formed by extending said wire mesh from both end portions of said insulation tube and by narrowing said extended wire mesh.

6. The noise measuring cable according to claim 5, wherein a connection component, which can be attached to or detached from a connection object, is connected to a tip portion of said extended portion.
